(19) United States
(12) Patent Application Publication
(10) Pub. No.: US 2004/0001818 A1
(43) Pub. Date: Jan. 1, 2004

(54) Methods of inhibiting angiogenesis using NADPH oxidase inhibitors

(75) Inventors: William C. Aird, Brookline, MA (US); Ruhul Abid, Belmont, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Beth Israel Deaconess Medical Center, Inc., Boston, MA (US)

(21) Appl. No.: 10/412,783

(22) Filed: Apr. 11, 2003

Related U.S. Application Data
(63) Continuation-in-part of application No. PCT/US01/31856, filed on Oct. 11, 2001.
(60) Provisional application No. 60/239,818, filed on Oct. 12, 2000.

(51) Int. Cl. A61K 38/44; A61K 31/135; A61K 31/12; A61K 31/03

(52) U.S. Cl. 424/94.4; 514/646; 514/751; 514/689

(57) ABSTRACT

Methods of inhibiting angiogenesis, endothelial cell migration or endothelial cell proliferation, by inhibiting NADPH oxidase inhibitors, production of reactive oxygen species, or by inhibition of mRNA induction of superoxide dismutase (e.g., mitochondrial SOD), are described herein.

### RELATED APPLICATIONS

This application is a continuation-in-part of International Application No. PCT/US01/31856, which designated the United States and was filed Oct. 11, 2001, published in English, which claims the benefit of U.S. Provisional Application No. 60/239,818, filed Oct. 12, 2000.

The entire teachings of the above applications are incorporated herein by reference.

### GOVERNMENT SUPPORT

The invention was supported, in whole or in part, by a grant 1P50 HL63609-01 from the National Institutes of Health. The Government has certain rights in the invention.

### BACKGROUND OF THE INVENTION

Superoxide and other reactive oxygen species (ROS) have been implicated in the initiation and progression of several pathophysiological states. At the same time, there is increasing evidence that ROS play an important role as signal transduction intermediates. For example, ROS have been shown to mediate cellular response to cytokines and growth factors and to induce changes in gene expression, cell differentiation, immune activation and apoptosis. Several intracellular sources contribute to the production of ROS, including xanthine oxidase, NADPH oxidase, endothelial nitric oxide synthase (eNOS), lipoxygenases, cyclooxygenases, and mitochrondrial respiration.

The superoxide dismutase (SOD) family includes cytosolic Cu,Zn-SOD, mitochondrial MnSOD and extracellular Cu,Zn-SOD (EC-SOD). By converting superoxide to H₂O₂ and O₂, these enzymes inhibit radical reactions leading to oxidative damage. MnSOD is the primary antioxidant defense against superoxide radicals within the mitochondria. Many studies have shown that increased cellular levels of MnSOD are cytoprotective against oxidative stress. Indeed, MnSOD expression is upregulated by a variety of pro-inflammatory mediators, including LPS, TNF-α, IL-1β, INF-gamma, α-thrombin, and ionizing radiation.

The importance of MnSOD in cell biology is evidenced by genetic mouse models in which the gene is either deleted or overexpressed. For example, mice that are null for the MnSOD gene are embryonic lethal and develop cardiomyopathy. On the other hand, targeted overexpression of MnSOD in transgenic mice is associated with the suppression of spontaneous apoptosis and protection against ischemic reperfusion injury in the heart.

Vascular endothelial growth factor (VEGF) is a potent vascular endothelial cell-specific mitogen that induces physiological angiogenesis in embryogenesis, in wound healing and in the reproductive tract. VEGF has also been implicated in pathological states of angiogenesis, including proliferative diabetic retinopathy, solid tumor growth, and collateral vessel formation in cardiovascular disease. In addition to its role in angiogenesis, VEGF has been shown to alter microvascular permeability and vasodilation, to inhibit apoptosis (Gerber, H. P. et al. (1998) J. Biol. Chem. 273:30336-43) and to promote cell migration.

### SUMMARY OF THE INVENTION

The present invention is based on the discovery that inhibition of NADPH oxidase inhibits proliferation and migration of endothelial cells and angiogenesis. As described herein, basal levels of NADPH oxidase-derived reactive oxygen species (ROS) are associated with VEGF signaling with respect to MnSOD induction. Also as described herein, endothelial cell proliferation was inhibited by VEGF and NADPH oxidase inhibitors, in particular the NADPH oxidase inhibitors (diphenyleneiodonium (DPI), apocynin or 4-(2-aminoethyl)-benzenesulfonyl fluoride (AEBSF)) and their effects on endothelial cell proliferation are described herein.

The present invention encompasses methods of inhibiting angiogenesis in a tissue, where the method comprises contacting the tissue with an inhibitor of NADPH oxidase, for example, a compound or a chemical, such as diphenyleneiodonium (DPI), apocynin, 4-(2-aminoethyl)-benzenesulfonyl fluoride (AEBSF) or an enzyme, such as a superoxide dismutase mitochondrial SOD (MnSOD), cytosolic Cu,Zn-SOD or extracellular Cu,Zn-SOD.

The present invention also encompasses methods of inhibiting angiogenesis in a tissue, where the method comprises inhibiting the production of reactive oxygen species (ROS) in the tissue, e.g., by contacting the tissue with an inhibitor of NADPH oxidase, e.g., a compound or a chemical, such as diphenyleneiodonium (DPI), apocynin, AEBSF, or an enzyme, such as a superoxide dismutase, mitochondrial SOD (MnSOD), cytosolic Cu,Zn-SOD or extracellular Cu,Zn-SOD. The inhibition of the production of reactive oxygen species (ROS) can also be accomplished by contacting the tissue with an inhibitor of superoxide dismutase (SOD) (e.g. mitochondrial SOD (MnSOD)) mRNA induction.

In another embodiment of the present invention methods of inhibiting angiogenesis in a tissue, where the method includes inhibiting induction of mRNA of a superoxide dismutase (SOD), for example, mitochondrial SOD (MnSOD), cytosolic Cu,Zn-SOD or extracellular Cu,Zn-SOD, in the tissue are provided.

The invention further comprises methods of inhibiting endothelial cell migration and proliferation by contacting the cells with an inhibitor of NADPH oxidase, such as a compound or a chemical, e.g., diphenyleneiodonium (DPI), apocynin, AEBSF, or an enzyme, e.g., a superoxide dismutase, e.g., mitochondrial SOD (MnSOD), cytosolic Cu,Zn-SOD or extracellular Cu,Zn-SOD or by inhibiting the production of reactive oxygen species (ROS) in the tissue, e.g., by contacting the tissue with an inhibitor of NADPH oxidase, e.g., a chemical, e.g., diphenyleneiodonium (DPI), apocynin, AEBSF, or an enzyme, e.g., a superoxide dismutase, e.g., mitochondrial SOD (MnSOD), cytosolic Cu,Zn-SOD or extracellular Cu,Zn-SOD. The inhibition of the production of reactive oxygen species (ROS) can also be accomplished by contacting the cells with an inhibitor of superoxide dismutase (SOD) (e.g. mitochondrial SOD (MnSOD)) mRNA induction.

The invention also features a method of inhibiting endothelial cell migration in a tissue, where the method includes inhibiting induction of mRNA of a superoxide dismutase (SOD), e.g., mitochondrial SOD (MnSOD), cytosolic Cu,Zn-SOD or extracellular Cu,Zn-SOD, in the tissue.

In a further aspect, the invention features a method of inhibiting endothelial cell proliferation in a tissue, where the method includes contacting the tissue with an inhibitor of NADPH oxidase, e.g., a chemical, e.g., diphenyleneiodonium (DPI), apocynin, AEBSF, or an enzyme, e.g., a superoxide dismutase, e.g., mitochondrial SOD (MnSOD), cytosolic Cu,Zn-SOD or extracellular Cu,Zn-SOD.

Another features of the invention is a method of inhibiting endothelial cell proliferation in a tissue, where the method includes inhibiting the production of reactive oxygen species (ROS) in the tissue, e.g., by contacting the tissue with an inhibitor of NADPH oxidase, e.g., a chemical, e.g., diphenyleneiodonium (DPI), apocynin, AEBSF, or an enzyme, e.g., a superoxide dismutase, e.g., mitochondrial SOD (MnSOD), cytosolic Cu,Zn-SOD or extracellular Cu,Zn-SOD. The inhibition of the production of reactive oxygen species (ROS) can also be accomplished by contacting the tissue with an inhibitor of superoxide dismutase (SOD) (e.g. mitochondrial SOD (MnSOD)) mRNA induction.

As described herein, inhibiting endothelial cell migration and proliferation can be accomplished by inhibiting induction of mRNA of a superoxide dismutase (SOD), e.g., mitochondrial SOD (MnSOD), cytosolic Cu,Zn-SOD or extracellular Cu,Zn-SOD.

The present invention also encompasses methods of inhibiting VEGF-mediated angiogenesis in a tissue, where the method includes contacting the tissue with an inhibitor of NADPH oxidase, e.g., a chemical, e.g., diphenyleneiodonium (DPI), apocynin, AEBSF, or an enzyme, e.g., a superoxide dismutase, e.g., mitochondrial SOD (MnSOD), cytosolic Cu,Zn-SOD or extracellular Cu,Zn-SOD.

In an additional aspect, the invention features a method of inhibiting VEGF-mediated angiogenesis in a tissue, where the method includes inhibiting the production of reactive oxygen species (ROS) in the tissue, e.g., by contacting the tissue with an inhibitor of NADPH oxidase, e.g., a compound or a chemical, such as diphenyleneiodonium (DPI), apocynin, AEBSF, or an enzyme, e.g., a superoxide dismutase, e.g., mitochondrial SOD (MnSOD), cytosolic Cu,Zn-SOD or extracellular Cu,Zn-SOD. The inhibition of the production of reactive oxygen species (ROS) can also be accomplished by contacting the tissue with an inhibitor of superoxide dismutase (SOD) mRNA induction.

The invention also features methods of inhibiting VEGF-mediated angiogenesis in a tissue, where the method includes inhibiting induction of mRNA of a superoxide dismutase (SOD) in the tissue.

The invention also features a composition that includes an inhibitor of NADPH oxidase (for example, a compound or a chemical, e.g., diphenyleneiodonium (DPI), apocynin, AEBSF, or an enzyme, e.g., a superoxide dismutase (SOD), e.g., mitochondrial SOD (MnSOD), cytosolic Cu,Zn-SOD or extracellular Cu,Zn-SOD) where the composition has one or more of the following properties: (a) the ability to inhibit angiogenesis, (b) the ability to inhibit endothelial cell migration, (c) the ability to inhibit endothelial cell proliferation, or (d) the ability to inhibit VEGF-mediated angiogenesis, where the composition can optionally include a pharmaceutically compatible carrier.

The invention also features a composition that includes an inhibitor of ROS production (e.g., a chemical, e.g., diphenyleneiodonium (DPI), apocynin, AEBSF, or an enzyme, e.g., a superoxide dismutase (SOD), e.g., mitochondrial SOD (MnSOD), cytosolic Cu,Zn-SOD or extracellular Cu,Zn-SOD) where the composition has one or more of the following properties: (a) the ability to inhibit angiogenesis, (b) the ability to inhibit endothelial cell migration, (c) the ability to inhibit endothelial cell proliferation, or (d) the ability to inhibit VEGF-mediated angiogenesis, where the composition can optionally include a pharmaceutically compatible carrier.

In additional aspects, the invention features the use of such compositions as described above in the preparation of a medicament for treating an angiogenesis-mediated disorder wherein the treatment involves inhibiting angiogenesis in a tissue, inhibiting endothelial cell migration in a tissue, inhibiting endothelial cell proliferation in a tissue, and/or inhibiting VEGF-mediated angiogenesis in a tissue. In particular, the disorder can be tumor growth, or cancer.

The invention also encompasses methods of inducing or enhancing angiogenesis in a tissue, comprising enhancing or inducing the expression of NADPH oxidase, or by increasing its activity and methods of inducing or enhancing angiogenesis in a tissue, comprising enhancing or inducing the production of ROS.

As a result of the work described herein, methods of inhibiting endothelial cell proliferation and migration using NADPH oxidase inhibitors are available. Importantly, when endothelial cell proliferation and migration are inhibited, angiogenesis is inhibited and thus, new methods of inhibiting angiogenesis and tumor growth are now available.

### BRIEF DESCRIPTION OF THE DRAWINGS

FIGS. 1A and 1B are a pair of Northern blots showing that VEGF induces MnSOD mRNA in human coronary artery endothelial cells (HCAECs) (FIG. 1A) and human pulmonary artery endothelial cells (HPAECs) (FIG. 1B).

FIGS. 2A and 2B are a pair of Northern blots. FIG. 2A shows VEGF-induced MnSOD mRNA production at 0, 1, 5, 10 and 100 ng/ml treatment with VEGF (FIG. 2A). FIG. 2B is a Northern blot showing MnSOD mRNA production in HCAECs treated with no actinomycin D nor VEGF, actinomycin D only, VEGF only, and both actinomycin D and VEGF.

FIGS. 3A and 3B, which are a pair of western blots showing induction of MnSOD protein in HPAECs (FIG. 3A) and HCAECs (FIG. 3B) cells, at 0, 0.5, 1, 2, 4, 12 and 24 hours after treatment with VEGF. As internal controls, the membranes were stripped and probed with anti-Egr-1 and β-actin antibodies.

FIG. 4 is a Northern blot showing induction of MnSOD in serum-starved HCAECs when treated with VEGF (50 ng/ml) in the presence of either DPI (5, 25 or 100 μM), apocynin (“anth”) (0.3, 2, 5 mM) or allopurinol (0.1, 0.5 mM).

FIGS. 5A, 5B, 5C and 5D are a set of four flow cytometry plots showing ROS generation in control untreated HCAECs (FIG. 5A), VEGF-treated HCAECs (FIG. 5B), DPI-treated HCAECs (FIG. 5C), and PMS-treated HCAECs (FIG. 5D).

FIG. 6 is a Northern blot showing the effect on VEGF-mediated induction of MnSOD mRNA production of BIM (bisindolylmaleimide I; “BIM”; 0.1, 1, 5 μM), PD98059 (“PD”; 5, 20, 100 μM) and Wortmannin (“WORT”; 10, 100 nM).

FIG. 7 is a histogram showing [³H]-thymidine incorporation (y-axis) in HCAECs that have been serum starved in 0.5% FBS in the absence (control) o presence of VEGF (V) and or inhibitors (D=DPI; A=anthrone) (x-axis) for 16 hours.

FIG. 8 is a histogram showing [³H]-thymidine incorporation (y-axis) in HCAECs that have been serum starved in 0.5% FBS in the absence (control) or presence of VEGF (V) and or inhibitors (D=DPI; A=anthrone) (x-axis) for 24 hours.

FIG. 9 is a histogram showing [³H]-thymidine incorporation (y-axis) in HCAECs grown in 0.5% FBS or 5.0% FBS in the absence (5.0% FBS) or presence of incremental doses of DPI (1, 5, 25, 40, 80 μM DPI) (x-axis).

FIG. 10 is a histogram showing [³H]-thymidine incorporation (y-axis) in HCAECs that have been grown in 10% FBS in the absence (C=control) or presence of inhibitors (DPI; Allo=allopurinol; L-NAME) (x-axis).

FIG. 11 is a histogram showing [³H]-thymidine incorporation (y-axis) in HCAECs that have been grown in 0.5% FBS or 5.0% FBS in the absence (5.0% FBS) or presence of incremental doses (300 μM, 500 μM, 1 mM, 2 mM, 3 mM) apocynin (x-axis).

FIG. 12 is a histogram showing thymidine incorporation (cpm, x-axis) in HCAECs treated with 5% FBS and 4-(2-aminoethyl)-benzenesulfonyl fluoride (AEBSF) at 0, 5, 50, 100 and 500 μM (y-axis). “0.5%” represents thymidine uptake in HCAECs treated with only 0.5% FBS. “*”=P<0.05 relative to control, untreated cells. “†”=P<0.05 relative tp treated cells.

FIG. 13 is a histogram showing [³H]-thymidine incorporation (y-axis) in mouse embryonic fibroblasts that have been grown in 0.5% FBS or 10% FBS in the absence (10% FBS) or presence of inhibitors (Apo=apocynin; allo=allopurinol; L-NAME) for 16 hours.

FIG. 14 is a histogram showing the migration in terms of cell count (y-axis) of HCAECs in a Boyden chamber. The cells were serum starved in 0.5% FBS and treated with either VEGF or DPT or VEGF and DPI (x-axis).

FIG. 15 is a histogram showing the migration in terms of cell count (y-axis) of HCAECs in a Boyden chamber. The cells were serum starved in 0.5% FBS and treated with either VEGF (“V”), VEGF+allopurinol (“V+allo”) or VEGF+AEBSF (“V+AEBSF”) (x-axis). “*”=P<0.05 relative to control, untreated cells. “†”=P<0.05 relative tp treated cells.

FIGS. 16A and 16B are a pair of histograms showing VEGF-mediated chemotaxis in control HCAECs (“C”), or HCAECs treated with 50 ng/ml VEGF in the absence or presence of increasing doses of DPI (0.5, 1.0, 5.0 or 10 μM; FIG. 16A) or AEBSF (5, 50, 100 or 250 μM; FIG. 16B). “*”=P<0.05 relative to control, untreated cells. “†”=P<0.05 relative tp treated cells.

FIGS. 17A, 17B and 17C are representations of Northern blots depicting regulation of VEGF-mediated MnSOD expression. PI3K inhibition by LY294002, AKT inhibition by DN-AKT or activation of forkhead by inhibition of phosphorylation (TM-FKHRL1) upregulates VEGF-mediated MnSOD expression. Northern blots of HCAEC cells that were grown to subconfluency (85-90%) and serum starved overnight before treatment. FIG. 17A shows serum starved cells that were pretreated with or without LY294002 (20 μM) for 30 min before incubation in the absence or presence of VEGF (50 ng/ml) for 4 hr. The blots were probed with radiolabeled MnSOD cDNA. FIG. 17B is a Northern blot derived from cells that were infected with replication-deficient adenoviruses expressing either βGal, DN-AKT (S473A/T308A) or CA-AKT (Gag) before serum starvation as indicated. FIG. 17C is a Northern blot derived from cells that were infected with adenoviruses expressing either βGal, TM-FKHRL1 (an active, phosphorylation-resistant, T32A/S253A/S315A, triple mutant) or WT-FKHRL1 (wild-type) forkhead isoform.

FIGS. 18A and 18B are Northern blots demonstrating PKCδ and PKCζ signaling pathways positively regulate VEGF-mediated MnSOD expression in endothelial cells. FIG. 18A shows HCAEC that were serum starved and pretreated with or without MEK-inhibitor (PD98509, 50 μM) or PKC-inhibitor (GF109203X, 1 μM) for 30 min before VEGF treatment. The Northern blots performed using MnSOD probe shows that inhibition of PKC, but not MAPK, abrogates VEGF-induced MnSOD expression. FIG. 18B shows HCAEC that were infected with adenovirus expressing either WT- or DN-PKCδ or WT- or DN-PKCζ isoforms. The cells were serum starved overnight, treated with or without VEGF for 4 hr. Total RNA was extracted and Northern blots were performed using a full-length radiolabeled probe of MnSOD.

### DETAILED DESCRIPTION OF THE INVENTION

The present invention is based on the discovery that inhibition of NADPH oxidase inhibits proliferation and migration of endothelial cells and angiogenesis. Specifically, inhibition of NADPH oxidase-derived reactive oxygen species (ROS), e.g., by NADPH oxidase inhibitors (e.g., diphenyleneiodonium (DPI), apocynin or 4-(2-aminoethyl)-benzenesulfonyl fluoride (AEBSF)) results in decreased VEGF signaling, as seen, e.g., by incorporation of [³H]-thymidine in VEGF-stimulated endothelial cells.

Mitochrondrial superoxide dismutase (MnSOD) is the primary antioxidant defense against reactive oxygen species (ROS) within the mitochondrial matrix. MnSOD converts the superoxide radical to H₂O₂, which is then scavenged by catalase and glutathione peroxidase. Vascular endothelial growth factor (VEGF) is a potent vascular endothelial cell-specific mitogen that modulates endothelial cell function. VEGF induces endothelial cell proliferation, and NADPH oxidase inhibitors (e.g., DPI, apocynin or AEBSF) abrogate this response, and also inhibit VEGF-mediated migration of endothelial cells, e.g., in a Boyden chamber. In addition, NADPH oxidase inhibitors interfere with serum-mediated induction of endothelial cell growth.

These findings show that endothelial cell proliferation and migration is dependent on NADPH-oxidase-derived ROS under both basal and stimulated conditions, and more importantly, inhibition of NADPH oxidase inhibits endothelial cell proliferation and migration under all conditions tested.

It is also shown herein that VEGF induces MnSOD protein and mRNA in human coronary and pulmonary artery endothelial (HCAE and HPAE) cells. VEGF-mediated induction of MnSOD mRNA was inhibited by pretreatment with the NADPH oxidase inhibitors, DPI, apocynin and AEBSF. Induction of MnSOD was unaffected by the addition of L-NAME or the xanthine oxidase inhibitor, allopurinol. The addition of DPI to endothelial cell cultures resulted in a significant reduction of ROS, as measured by the oxidation of 2′,7′-dichlorodihydrofluorescein, while the addition of prooxidants had the opposite effect. In contrast, VEGF treatment failed to induce detectable changes in ROS. These findings show that VEGF-mediated induction of MnSOD in endothelial cells is dependent upon ambient levels of NADPH oxidase-generated ROS, and that VEGF induces MnSOD expression and that VEGF signaling requires a threshold level NADPH oxidase-generated ROS.

While ROS have traditionally been viewed as cytotoxic molecules, they are now recognized to play a critical role in signal transduction and transcriptional regulation in several types of cells, including endothelial cells (Bouloumie, A. et al. (1999) FASEB J. 13:1231-8; Rahman, A. et al. (1998) Am. J. Physiol. 275(3, Pt 1):L533-44; Weber, C. et al. (1994) Arterioscler. Thromb. 14:1665-73; Zachary, I. (1998) Exp. Nephrol. 6:480-7; Lopez-Ongil, S. et al. (2000) J. Biol. Chem. 275:26423-7). For example, ROS have been implicated in TNF-α-mediated induction of VCAM-1 and E-selectin within the endothelium. Recent studies have demonstrated an important role for ROS in shear stress-induced phosphorylation of ERK1/2 in endothelial cells. In addition, ROS have been shown to increase expression and/or DNA-binding of transcription factors such as NFκβ and API (Becker, L. B. et al. (1999) Am. J. Physiol. 2777 (6, Pt 2):H2240-6; Chen, Z, et al. (1998) J. Mol. Cell. Cardiol. 30:2281-9; Das, D. K. et al. (1993) Cardiovasc. Res. 27:578-84; Hashimoto, E. et al. (1994) Am. J. Physiol. 267(5 Pt. 2):H1948-54).

As described herein, VEGF induces MnSOD expression by an NADPH oxidase-dependent mechanism. NADPH oxidase has been previously implicated in endothelial cell signaling. Various components of the leukocyte NADPH oxidase complex have been identified in endothelial cells, including gp91hox, p47phox and p22phox (Gorlach, A. et al. (2000) Circ. Res. 87:26-32; De Keulenaer, G. W. et al. (1998) Circ. Res. 82:1094-101; Wei, Z. et al. (1999) Circ. Res. 85:682-9). Moreover, a number of studies have provided evidence for the role of NADPH oxidase as the primary determinant of basal ROS generation in the endothelium. Finally, temporal changes in NADPH oxidase activity and secondary increases in ROS production have been reported in studies of endothelial cells exposed to oscillatory and steady state shear stress (Zhao, G. et al. (1997) Am. J. Physiol. 273(6 Pt 1):L1112-7), the cessation of blood flow over flow-adapted endothelial cells (Al-Mehdi, A. B. et al. (1998) Circ. Res. 83:730-7), ischemia (Lander, H. M. (1997) FASEB J. 11:118-24; Kunsch, C. et al. (1999) Circ. Res. 85:753-66) and high concentrations of K⁺ (Kunsch, C. et al. (1999) Circ. Res. 85:753-66).

Although the results described herein show a link between VEGF and NADPH oxidase activity, the failure of the growth factor to directly shift the fluorescence distribution of endothelial cells loaded with 2′,7′-dichlorofluorescein diacetate (DCFDA) argue against a direct effect of VEGF on ROS production. Since DCF fluorescence measures gross changes in ROS production, it cannot formally be ruled out that VEGF has a highly localized effect on reactive species. However, the results disclosed herein support a model in which VEGF-mediated signaling is dependent upon ambient levels of ROS. The finding that inhibition of NADPH oxidase abrogated the response of MnSOD to VEGF, while inhibitors of xanthine oxidase and NOS had no such effect, is consistent with the established role of NADPH oxidase as the primary determinant of basal ROS in endothelial cells. In a recent study, TNF-α-mediated induction of ICAM-1 in endothelial cells was also shown to depend on an ambient flux of NADPH oxidase-derived ROS rather than their net incremental generation. Together with the results presented herein, these results suggest that NADPH oxidase contributes to a net oxidant state in endothelial cells and that constitutive levels of ROS are critical components of signal transduction networks.

One other study has examined the effect of VEGF on ROS production. Zachary et al. ((1998) Exp. Nephrol. 6:480-7) studied VEGF-stimulated ROS production in bovine retinal microvascular endothelial cells. The response was abrogated by the addition of SOD, L-NAME and by the peroxynitrite scavenger, urate (Banai, S. et al. (1994) Cardiovasc. Res. 28:1176-9). These findings supported an important role for NOS in mediating VEGF-induced changes in ROS. In the present study, however, a role for NOS in VEGF regulation of MnSOD could not be confirmed. Based on these differences, it is proposed that the effect of VEGF on ROS generating pathways can vary between species and/or different sites of the vascular tree.

The link between VEGF signaling and MnSOD expression could have important biological implications. First, ROS have been shown to induce mitochondrial damage and dysfunction, leading to impaired function of Krebs' citric acid cycle and activation of apoptotic pathways. Since MnSOD catalyzes the removal of O₂⁻, the enzyme has the potential to enhance cell survival. Indeed, it is tempting to speculate VEGF-mediated induction of MnSOD represents an important mechanism by which the growth factor exerts its anti-apoptotic effects. Second, the increased SOD activity is predicted to shift the balance between intracellular levels of O₂⁻ and H₂O₂. Various reactive species play different roles in signaling. For example, O₂⁻ but not H₂O₂ increases ECE expression in endothelial cells, while H₂O₂ and not O₂⁻ has been linked to increased levels of eNOS. It follows from these observations that increased ratios of H₂O₂:O₂⁻ can contribute to the specificity of downstream signal transduction pathways (Li, J. et al. (1996) Am. J. Physiol. 270(5 Pt 2):H1803-11). Third, increased levels of MnSOD and perhaps other SOD can protect the endothelial cell from VEGF-mediated changes in peroxynitrite (ONOO⁻) formation. VEGF has been shown to induce NO activity in endothelial cells (Kuroki, M. et al. (1996) J. Clin. Invest. 98:1667-75). Newly generated NO can react with O₂⁻ to produce peroxynitrite (ONOO⁻), leading to endothelial cell dysfunction and mitochondrial damage (Chua, C. C. et al. (1998) Free Radic. Biol. Med. 25:891-7; Bouloumie, A. et al. (1999) Cardiovasc. Res. 41:773-80). SOD competes with NO for scavenging of O₂⁻, thereby inhibiting the production of ONOO⁻ and increasing the bioavailability of NO. Based on the results disclosed herein, VEGF-mediated induction of SOD can serve to offset the ROS-generating capacity of elevated NO. In other words, the co-regulation of SOD and eNOS can serve to reduce the prooxidant potential of NO and to divert NO activity to biologically important functions. Finally, the possibility that VEGF induces local changes in ROS that are below the limits of detection of these assays cannot be excluded. If this were the case, local production of O₂⁻ might serve to upregulate MnSOD levels, which would then attenuate further ROS production and protect against cytotoxicity.

The present invention includes the method of treating an angiogenesis-mediated disease by inhibiting endothelial cell migration and/or proliferation. Methods of treatment include methods of inhibiting the activity of NADPH oxidase, and/or by inhibiting the production of ROS, and/or by preventing the induction of SOD (e.g., MnSOD) mRNA. In particular, endothelial cells can be contacted with the inhibitors described herein, where contact results in the inhibition of endothelial cell proliferation and/or migration.

The present invention also includes compositions that can be used for this purpose. Such compositions contain inhibitors of NADPH oxidase. Any NADPH oxidase can be used in the methods and compositions described herein. In particular, inhibitors such as DPI, apocynin, 4-(2-aminoethyl)-benzenesulfonyl fluoride (AEBSF) and MnSOD are encompassed by the present invention. Other inhibitors suitable for use in the present invention can be prepared by processes known to those of skill in the art. Such inhibitors can be tested in the assays described herein to determine their NADPH oxidase inhibiting activity. The design of such inhibitors can be based on chemical structure that results in a level of inhibiting activity comparable to the inhibitors described in the Examples herein. Such a composition can contain a effective amount of the inhibitor, or an agonist. “Agonist” means a molecule that mimics the activity of an inhibitor described herein having the ability to inhibit NADPH oxidase, and/or the production of ROS, which results in the inhibition of endothelial cell migration, proliferation or angiogenesis. Such agonists can be analogs of the inhibitors described herein, with one or more modifications. An agonist is not required to have precisely the same level of activity as the compound that it mimics, but can have increased or decreased activity, so long as it is capable of use. An effective amount of inhibitor, or agonist, as used herein, means that the inhibitor or agonist is administered in an amount sufficient to inhibit NADPH oxidase actively, which results in inhibition of endothelial cell proliferation and/or migration or angiogenesis in a tissue.

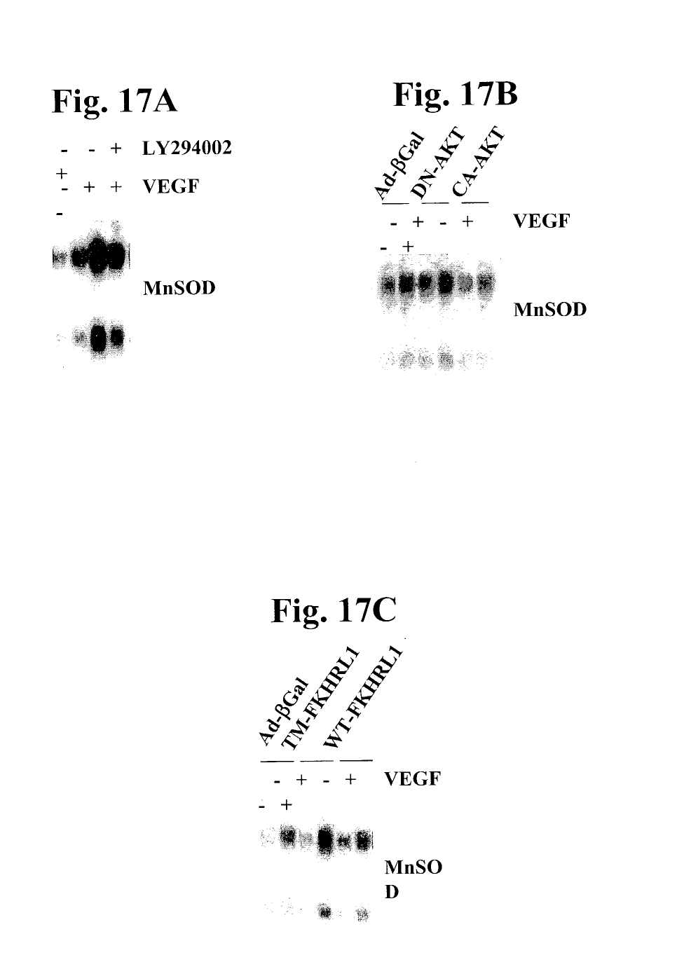

Disclosed herein are examples of VEGF-mediated induction of MnSOD in human coronary artery endothelial cells (HCAEC; FIGS. 17A-C, 18A and 18B). As VEGF induces MnSOD mRNA expression in human coronary artery endothelial cells (HCAEC) by a Rac1-regulated NADPH oxidase-dependent mechanism, further dissection of the signaling pathways involved in mediating VEGF stimulation of MnSOD identifies regulators of MnSOD expression. In the absence of VEGF, preincubation of HCAEC with PI3K inhibitors (20 μM LY294002 or 100 nM wortmannin) resulted in increased basal expression of MnSOD. In the presence of VEGF, PI3K inhibition resulted in superinduction of MnSOD mRNA (FIGS. 17A-C).

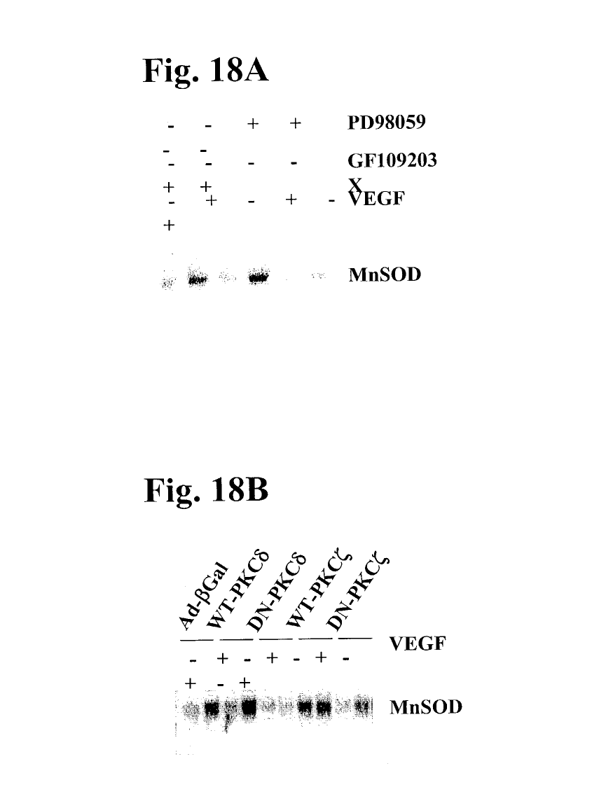

Additionally, adenovirus-mediated expression of a dominant negative AKT gene, DN-AKT (with the S473A, T308A substitution), or an activated form of forkhead (TM-FKHRL1) in HCAEC resulted in increased VEGF-stimulated expression of MnSOD, supporting a role for the AKT/forkhead pathway in mediating the inhibitory effect of PI3K. To elucidate the positive regulatory pathway(s) involved in mediating VEGF-induced MnSOD expression, HCAEC were pre-incubated with a variety of chemical inhibitors. VEGF stimulation of MnSOD was significantly reduced by 1 μM GF-109203X (PKC inhibitor), but not by 50 μM PD98059 (MAPK inhibitor) or 20 μM SB 203580 (p38 inhibitor), suggesting a positive role for protein kinase C (PKC) signaling. To determine the role of specific isoform(s) of PKC in this pathway, dominant negative (DN) PKC isoforms in HCAEC using adenovirus-mediated vectors were used (FIGS. 18A and 18B). DN-PKC δ and DN-PKC ζ abrogated VEGF-mediated induction of MnSOD, whereas DN-PKC α and DN-PKC ε had no such effect. Taken together, these results show that VEGF-mediated induction of MnSOD in HCAEC is negatively regulated by PI3K signaling pathways and positively regulated by novel and atypical PKC isoforms.

Angiogenesis-mediated diseases include, but are not limited to, cancers, solid tumors, blood-born tumors (e.g., leukemias), tumor metastasis, benign tumors (e.g., hemangiomas, acoustic neuromas, neurofibromas, trachomas, and pyogenic granulomas), rheumatoid arthritis, psoriasis, ocular angiogenic diseases (e.g., diabetic retinopathy, retinopathy of prematurity, macular degeneration, corneal graft rejection, neovascular glaucoma, retrolental fibroplasia, rubeosis), Osler-Webber Syndrome, myocardial angiogenesis, plaque neovascularization, telangiectasia, hemophiliac joints, angiofibroma, and wound granulation. The methods and compositions of the invention would also be useful in the treatment of diseases of excessive or abnormal stimulation of endothelial cells. These diseases include, but are not limited to, intestinal adhesions, Crohn's disease, atherosclerosis, scleroderma, and hypertrophic scars (i.e., keloids). The methods and compositions of the invention can be used as a birth control agent by preventing vascularization required for embryo implantation. The methods and compositions are also useful in the treatment of diseases that have angiogenesis as a pathologic consequence such as cat scratch disease (Rochele minalia quintosa) and ulcers (Heliobacter pylori). The invention can also be used to prevent dialysis graft vascular access stenosis, and obesity, e.g., by inhibiting capillary formation in adipose tissue, thereby preventing its expansion. The methods and compositions can also be used to treat localized (e.g., nonmetastisized) diseases.

Alternatively, where an increase in angiogenesis is desired, e.g., in wound healing, or in post-infarct heart tissue, an antagonist, antibodies or antisera to the compositions can be used to block localized, native anti-angiogenic processes, and thereby increase formation of new blood vessels so as to inhibit atrophy of tissue.

The methods and compositions of the present invention can be used in combination with other compositions and procedures for the treatment of diseases. For example, a tumor can be treated conventionally with surgery, radiation, chemotherapy, or immunotherapy, combined with methods and compositions of the present invention. The methods and compositions of the present invention can then also be subsequently administered to the patient to extend the dormancy of micrometastases and to stabilize and inhibit the growth of any residual primary tumor. The compositions or agonists thereof, or combinations thereof, can also be combined with other anti-angiogenic compounds, or proteins, fragments, antisera, receptor agonists, receptor antagonists of other anti-angiogenic proteins (e.g., angiostatin, endostatin, etc.). Additionally, the compositions of the present invention, and/or agonists or antagonists thereof are combined with pharmaceutically acceptable excipients, and optionally sustained-release matrix, such as biodegradable polymers, to form therapeutic compositions. The compositions of the present invention also can contain other anti-angiogenic compounds, such as endostatin, angiostatin, and mutants, fragments, and analogs thereof. The compositions may further contain other agents that either enhance the activity of the inhibitor or compliment its activity or use in treatment, such as chemotherapeutic or radioactive agents. Such additional factors and/or agents may be included in the composition to produce a synergistic effect with the inhibitor of the invention, or to minimize side effects. Additionally, administration of the composition of the present invention may be administered concurrently with other therapies, e.g., administered in conjunction with a chemotherapy or radiation therapy regimen.

Pharmaceutical compositions can be made containing such compounds. Administration of such pharmaceutical compositions can be carried out in a variety of conventional ways known to those of ordinary skill in the art, such as oral ingestion, inhalation, topical or transdermal application, or intracranial, intracerebroventricular, intracerebral, intravaginal, intrauterine, oral, rectal or parenteral (e.g., intravenous, intraspinal, subcutaneous or intramuscular) route, or cutaneous, subcutaneous, intraperitoneal, parenteral or intravenous injection.

The compositions can be administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent, i.e., carrier or vehicle.

Modes of administration of such compositions include intravenous, intramuscular, intraperitoneal, intrasternal, subcutaneous and intraarticular injection and infusion. Pharmaceutical compositions for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (e.g., glycerol, propylene glycol, polyethylene glycol and the like), carboxymethylcellulose and suitable mixtures thereof, vegetable oils (e.g., olive oil) and injectable organic esters such as ethyl oleate. Proper fluidity may be maintained, for example, by the use of coating materials such as lecithin, by the maintenance of the required particle size in the case of dispersions and by the use of surfactants. These compositions may also contain adjuvants such as preservatives, wetting agents, emulsifying agents and dispersing agents. Prevention of the action of microorganisms may be ensured by the inclusion of various antibacterial and antifungal agents such as paraben, chlorobutanol, phenol sorbic acid and the like. It may also be desirable to include isotonic agents such as sugars, sodium chloride and the like. Prolonged absorption of the injectable pharmaceutical form may be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations may be sterilized, for example, by filtration through a bacterial-retaining filter or by incorporating sterilizing agents in the form of sterile solid compositions that can be dissolved or dispersed in sterile water or other sterile injectable media just prior to use.

Potential pharmaceutical compositions include those suitable for oral, rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural) administration. The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations suitable for parenteral administration include aqueous and non-aqueous sterile injection solutions, which can contain anti-oxidants, buffers, bacteriostats and solutes that render the formulation isotonic with the blood of the intended recipient; and aqueous and non-aqueous sterile suspensions that can include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

When a therapeutically effective amount of a pharmaceutical composition of the present invention is administered orally, the composition will be in the form of a tablet, capsule, powder, solution or elixir. When administered in tablet form, the pharmaceutical composition may additionally contain a solid carrier such as a gelatin or an adjuvant. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol.

When a therapeutically effective amount of composition of the present invention is administered by intravenous, cutaneous or subcutaneous injection, the composition of the present invention will be in the form of a pyrogen-free, parenterally acceptable aqueous solution. The preparation of such parenterally acceptable solutions, having due regard to pH, isotonicity, stability, and the like, is within the skill in the art. A preferred pharmaceutical composition for intravenous, cutaneous, or subcutaneous injection should contain, in addition to the compound of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

A composition of the present invention can be combined with a pharmaceutically acceptable carrier. Such a composition may also contain diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration.

The therapeutic compositions of the present invention can include pharmaceutically acceptable salts of the components therein, e.g., which may be derived from inorganic or organic acids. By “pharmaceutically acceptable salt” is meant those salts that are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge, et al., describe pharmaceutically acceptable salts in detail in J. Pharmaceutical Sciences (1977) 66:1 et seq., which is incorporated herein by reference in its entirety. Pharmaceutically acceptable salts include the acid addition salts that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. The salts may be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptonoate, hexanoate, fumarate, hydrochloride, hydrobromide, hydroiodide, 2-hydroxymethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl, and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids that can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

The compositions of the present invention may further contain other agents that either enhance the activity of the active ingredient of the composition or compliment their activity or use in treatment, such as chemotherapeutic or radioactive agents. Such additional factors and/or agents may be included in the composition to produce a synergistic effect with composition of the invention, or to minimize side effects. Additionally, administration of the composition of the present invention may be administered concurrently with other therapies, e.g., administered in conjunction with a chemotherapy or radiation therapy regimen.

As used herein, the terms “pharmaceutically acceptable,” “physiologically tolerable” and grammatical variations thereof as they refer to compositions, carriers, diluents and reagents, are used interchangeably and represent that the materials are capable of administration to or upon a mammal with a minimum of undesirable physiological effects such as nausea, dizziness, gastric upset and the like. The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions, in liquid prior to use can also be prepared. The preparation can also be emulsified.

The active ingredient can be mixed with excipients that are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like that enhance the effectiveness of the active ingredient.

By “contacting” is meant not only topical application, but also those modes of delivery that introduce the composition into the tissues, or into the cells of the tissues.

Use of timed release or sustained release delivery systems are also included in the invention. Such systems are highly desirable in situations where surgery is difficult or impossible, e.g., patients debilitated by age or the disease course itself, or where the risk-benefit analysis dictates control over cure. The compound may be incorporated into biodegradable polymers allowing for sustained release of the compound, the polymers being implanted in the vicinity of where drug delivery is desired, for example, at the site of a tumor or implanted so that the compound is slowly released systemically. Osmotic minipumps may also be used to provide controlled delivery of high concentrations of the compound through cannulae to the site of interest, such as directly into a metastatic growth or into the vascular supply to that tumor. The biodegradable polymers and their use are described, for example, in detail in Brem et al. (1991) (J. Neurosurg. 74:441-446), which is hereby incorporated by reference in its entirety.

A sustained-release matrix, as used herein, is a matrix made of materials, usually polymers, which are degradable by enzymatic or acid/base hydrolysis or by dissolution. Once inserted into the body, the matrix is acted upon by enzymes and body fluids. The sustained-release matrix desirably is chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The compositions of the present invention can be in the form of a liposome in which the inhibitor of the present invention (or agonist thereof) is combined, in addition to other pharmaceutically acceptable carriers, with amphipathic agents such as lipids that exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

A pharmaceutical composition of the present invention may be a solid, liquid or aerosol and may be administered by any known route of administration. Examples of solid compositions include pills, creams, and implantable dosage units. The pills may be administered orally, the therapeutic creams may be administered topically. The implantable dosage unit can be administered locally, for example at a tumor site, or can be implanted for systemic release of the angiogenesis-modulating composition, for example subcutaneously. Examples of liquid composition include formulations adapted for injection subcutaneously, intravenously, intraarterially, and formulations for topical and intraocular administration. Examples of aerosol formulation include inhaler formulation for administration to the lungs.

The inhibitors of the present invention, or agonists thereof, can also be included in a composition comprising a prodrug. As used herein, the term “prodrug” refers to compounds that are rapidly transformed in vivo to yield the parent compound, for example, by enzymatic hydrolysis in blood. A thorough discussion is provided in T. Higuchi and V. Stella, Prodrugs as Novel Delivery Systems, Vol. 14 of the ACS Symposium Series and in Edward B. Roche, ed., Bioreversible Carriers in Drug Design, American Pharmaceutical Association and Pergamon Press, 1987, both of which are incorporated herein by reference. As used herein, the term “pharmaceutically acceptable prodrug” refers to (1) those prodrugs of the compounds of the present invention that are, within the scope of sound medical judgement, suitable for use in contact with the tissues of humans and animals without undue toxicity, irritation, allergic response and the like, commensurate with a suitable benefit-to-risk ratio and effective for their intended use and (2) zwitterionic forms, where possible, of the parent compound.

### EXAMPLES

### Example 1

Materials and Methods Used Herein

Mice and injection of growth factors. Female FVB mice (4 to 8 weeks old; 18 to 22 grams) were obtained from Taconic (Germantown, N.Y., USA). All protocols were approved by the Institutional Animal Care and Use Committee of the Beth Israel Deaconess Medical Center. Mice were injected intraperitoneally with VEGF (1.0 μg/g body weight).

Cell culture. Human coronary artery endothelial cells (HCAECs) (Clonetics Corporation, San Diego, Calif., USA) and human pulmonary artery endothelial cells (HPAECs) were grown in Endothelial Growth Medium-2-MV (EGM-2-MV) BulletKit (Clonetics Corporation, San Diego, Calif., USA). At 80-90% confluence, the cells were serum-starved in DMEM containing 0.5% FBS for 24 hours and subsequently treated with VEGF-supplemented medium for the times indicated. Human recombinant VEGF was purchased from PeproTech, Inc. (Rocky Hill, N.J., USA). In inhibition studies, the serum-starved HCAEC cells were preincubated with diphenyleneiodonium (DPI), apocynin, allopurinol, L-NAME, or anthrone for one hour and then incubated in the absence or presence of 40 ng/ml VEGF for another hour. In other studies, the serum-starved cells were pretreated with the MEK inhibitor PD98059 at a final concentration of 50 μM or a specific PKC inhibitor bisindolylmaleimide I (BIM; CalBiochem, San Diego, Calif., USA) at a final concentration of 5 μM for 30 minutes prior to addition of growth factors. An equal amount of DMSO was added to the control plates. Northern blot analyses. Ten micrograms total RNA was loaded on a 0.7% formaldehyde-containing agarose gel. The RNA was transferred to nylon membrane, covalently cross-linked with UV radiation, prehybridized for 6 hours, and hybridized for 18 hours at 42° C. with a [³²P]dCTP-labeled cDNA probe containing MnSOD or eNOS cDNA sequence. The membranes were subsequently stripped and probed with a radiolabeled 18S-ribosome probe. For actinomycin D experiments, HCAEC cells were pretreated with 10 μg/ml actinomycin D (Sigma Chemical Company, St. Louis, Mo., USA) for 30 minutes before addition of VEGF and were harvested for total RNA one hour later.

Western blot analyses. Whole cell protein extracts were prepared from serum-starved HCAECs and HPAECs one hour following addition of growth factor-supplemented or mock-treated media. Cells were washed twice with cold phosphate-buffered saline, harvested with a cell scraper, and lysed in ice-cold lysis buffer containing 20 mM HEPES, pH 7.5, 150 mM NaCl, 1% NP-40, 0.1% SDS, 1 mM EDTA, 1 mM DTT, 0.5 mM phenylmethylsulfonyl fluoride (PMSF), and the protease inhibitor cocktail (Boehringer Mannheim, Mannheim, Germany) for one hour. The resulting lysates were centrifuged at 10,000× g for 20 minutes and the supernatants were saved as whole cell protein extracts. Forty micrograms of protein were separated by 10% SDS-PAGE and electrotransferred to nitrocellulose membranes. Membranes were blocked with 5% nonfat dry milk in Tris-buffered saline with 0.1% Tween 20 for one hour at room temperature. The blot was incubated with primary rabbit polyclonal anti-MnSOD IgG (1:1000 dilution) (Santa Cruz Biotechnology Inc., Santa Cruz, Calif., USA) overnight at 4° C., followed by secondary antibody goat-anti-rabbit horseradish peroxidase conjugate (1:1000 dilution) (Pierce, Rockford, Ill., USA). The blot was washed extensively between each incubation step. Peroxidase activity was visualized with an enhanced chemiluminescense substrate system (Amersham, Arlington Heights, Ill., USA). Membranes were stripped and probed for eNOS and β-actin. Flow cytometry. Endothelial cells were labeled with DCFDA and oxidation of DCF was measured by flow cytometry.

### Example 2

Systemic Administration of VEGF Results in Upregulation of MnSOD.

Injection of VEGF into the intraperitoneal cavity of mice results in widespread distribution of the growth factor and phosphorylation of the VEGF receptor, Flk-1. In this study, therefore, organs were removed from mice four hours following intraperitoneal injection of either normal saline or 20 μg VEGF and harvested for total RNA. Northern blot analyses were then carried out to determine whether VEGF induced the expression of a panel of genes, including MnSOD. The systemic delivery of VEGF resulted in increased MnSOD mRNA levels in the heart and the kidney, but not in other tissues, including the lung, brain, liver, spleen and skeletal muscle. This result was reproduced on three separate occasions and raised the interesting possibility that MnSOD was induced by VEGF in a vascular bed-specific manner. Immunohistochemical assays did not identify the site of MnSOD induction in the heart. However, the in vivo results provided the necessary foundation for the cell culture studies described below.

### Example 3

VEGF Induces MnSOD mRNA in Cultured Endothelial Cells.

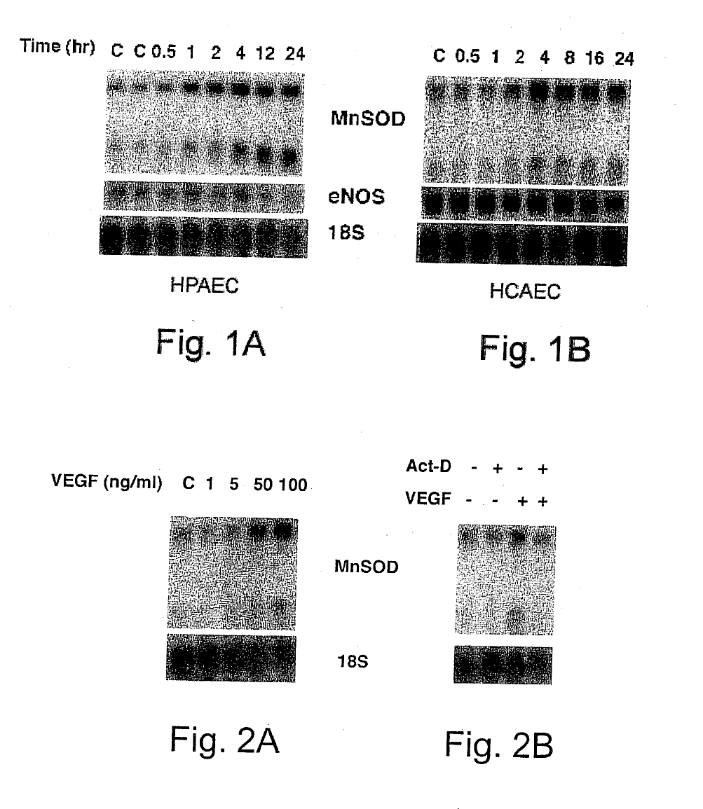

To determine whether VEGF induced expression of MnSOD in primary endothelial cell cultures, HCAECs and HPAECs were serum starved for 24 hours, treated with 40 ng/ml VEGF for 0, 0.5, 1, 2, 4, 12 and 24 hours, and then harvested for total RNA. Northern blots were performed and probed for human MnSOD. The results are shown in FIGS. 1A and 1B, which are a pair of Northern blots showing that VEGF induces MnSOD mRNA in human coronary artery endothelial cells (FIG. 1A) and human pulmonary artery endothelial cells (FIG. 1B). In response to VEGF, MnSOD mRNA levels were increased between one hour and 24 hours in HCAEC and between 2 and 24 hours in HPAEC, with peak levels occurring at 4 hours. The membranes were then stripped and reprobed for eNOS. As shown in FIGS. 1A and 1B (eNOS band), eNOS mRNA was not induced in either cell type by the administration of VEGF. This latter result is at odds with a previously published report showing VEGF-mediated induction of eNOS in human umbilical vein endothelial cells. In dose-response studies, MnSOD induction by VEGF was time dependent, with maximal induction occurring at 100 ng/ml VEGF. This is shown in FIG. 2A, which is a Northern blot showing VEGF-induced MnSOD mRNA production at 0, 1, 5, 10 and 100 ng/ml treatment with VEGF.

Finally, to determine whether VEGF-mediated induction of MnSOD was dependent upon new mRNA synthesis, HCAEC were pretreated with 10 μg/ml actinomycin D for 30 minutes prior to a 60 minute incubation with VEGF. The results are shown in FIG. 2B, which is a Northern blot showing MnSOD mRNA production in HCAECs treated with neither actinomycin D nor VEGF, actinomycin D only, VEGF only, and both actinomycin D and VEGF. Actinomycin D completely abolished VEGF-mediated induction of MnSOD mRNA, indicating that MnSOD induction by VEGF requires de novo mRNA synthesis.

### Example 4

VEGF Induces MnSOD Protein in Cultured Endothelial Cells.

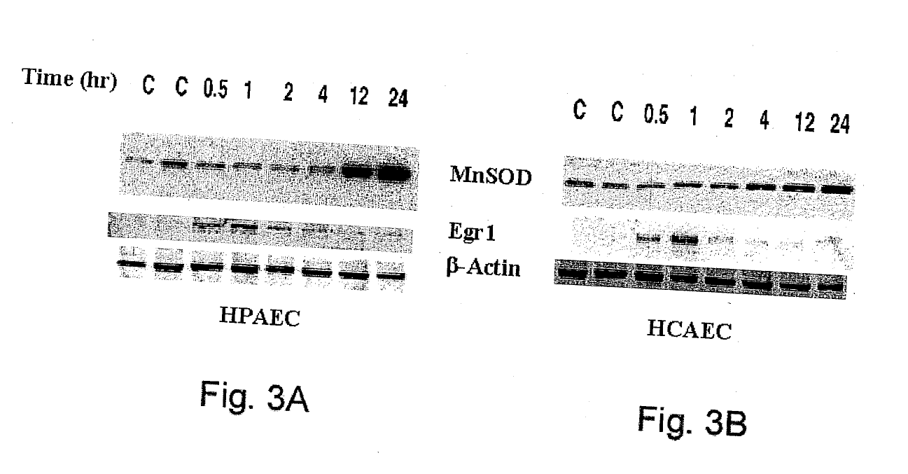

Serum-starved HCAECs and HPAECs were treated with 40 ng/ml VEGF for 0.5-24 hours and then harvested for total protein. The results are shown in FIGS. 3A and 3B, which are a pair of western blots showing induction of MnSOD protein in HPAEC (FIG. 3A) and HCAEC (FIG. 3B) cells, at 0, 0.5, 1, 2, 4, 12 and 24 hours after treatment with VEGF.

In Western blot analyses, MnSOD protein was significantly induced in HCAECs and HPAECs, with maximal levels occurring at 12-24 hours. As an internal control, the membranes were stripped and probed with an anti-Egr-1 antibody. VEGF-mediated induction of Egr-1 occurred at an earlier time point (between 0.5 and 1 hour). In contrast, VEGF did not increase eNOS protein levels at any time point. Taken together, these studies suggest that VEGF induces MnSOD both at a protein and mRNA level.

### Example 5

VEGF-Mediated Induction of MnSOD is Abrogated by NADPH Oxidase Inhibitors.

To determine whether the response of MnSOD to VEGF was mediated by ROS, HCAECs were serum starved and then treated with VEGF in the absence or presence of inhibitors of NADPH oxidase (DPI, apocynin), xanthine oxidase (allopurinol) and nitric oxide synthase (L-NAME).

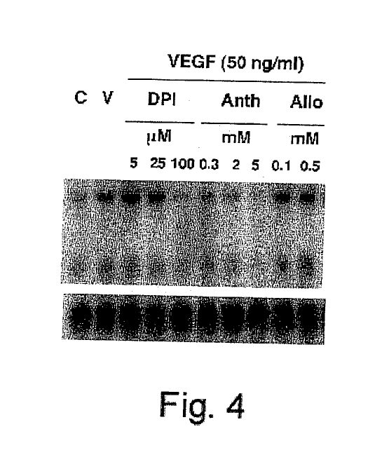

The results are shown in FIG. 4, which is a Northern blot showing induction of MnSOD in serum-starved HCAECs when treated with VEGF (50 ng/ml) in the presence of either DPI (5, 25 or 100 μM), apocynin (“anth”) (0.3, 2, 5 mM) or allopurinol (0.1, 0.5 mM). VEGF-mediated induction of MnSOD at four hours was inhibited by DPI and apocynin but not allopurinol or L-NAME (not shown). These results imply that the induction of MnSOD by VEGF is dependent on NADPH oxidase activity.

### Example 6

VEGF Does Not Increase ROS in Human Coronary Artery Endothelial Cells.

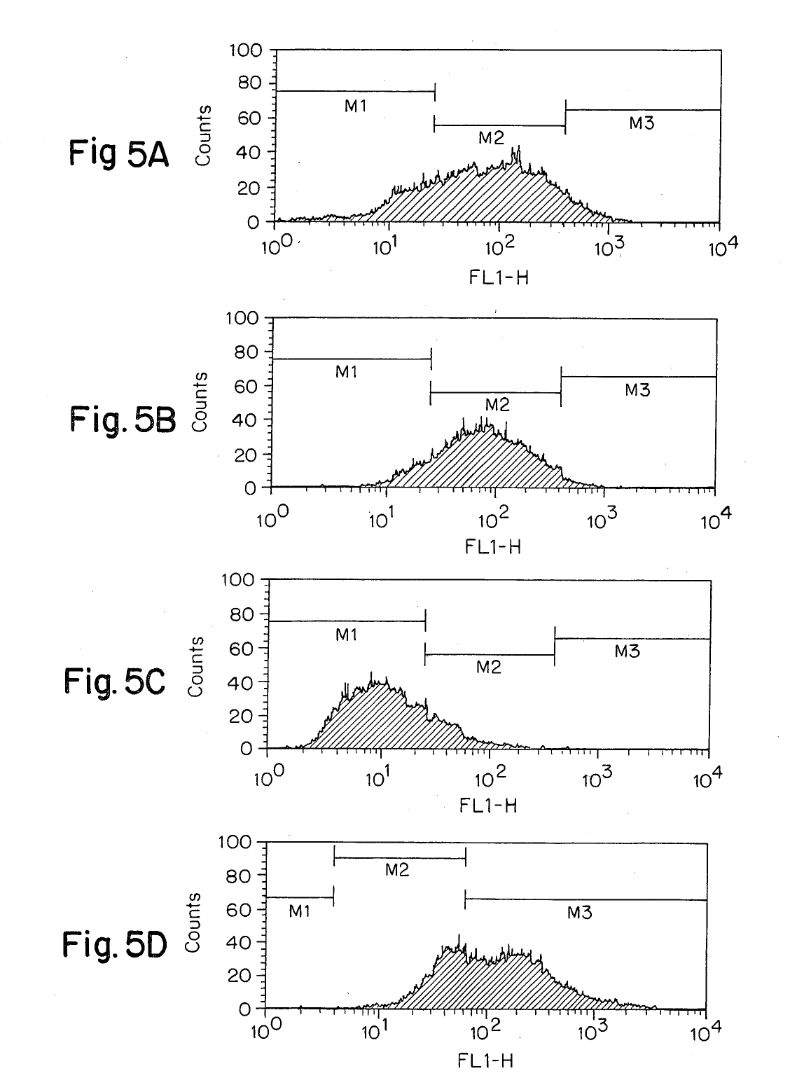

The above results raised the possibility that VEGF-mediated induction of MnSOD was dependent on increased NADPH oxidase activity and secondary ROS production. To test whether VEGF increases ROS production in endothelial cells, HCAECs were labeled with cells were labeled with DCFDA and oxidation of DCF was measured by flow cytometry. The results are shown in FIGS. 5A, 5B, 5C and 5D, which are a set of four flow cytometry plots showing ROS generation in untreated human coronary artery endothelial cells (control) (FIG. 5A), VEGF-treated HCAECs (FIG. 5B), DPI-treated HCAECs (FIG. 5C), and PMS-treated HCAECs (FIG. 5D). Control cells displayed a basal rate of DCF oxidation as indicated by the fluorescence distribution (FIG. 5A). VEGF-treated HCAECs also exhibited no change. Baseline oxidation of the fluorophore was significantly inhibited by the addition of DPI, as shown by the shift to the left in FIG. 5C, indicating reduced ROS production. Treatment with PMS showed a shift to the right (FIG. 5D), indicating increased ROS production. PMS is known to induce free oxygen radicals. PMA also significantly increased DCF oxidation. Allopurinol and L-NAME had no effect. These results suggest that basal or ambient levels of ROS in HCAEC are generated by the NADPH oxidase enzyme complex. The above findings support the notion that VEGF does not induce ROS in HCAEC, but rather signals through pathways that are dependent on ambient levels of NADPH-derived ROS.

### Example 7

VEGF-Mediated Induction of MnSOD is Mediated by a PKC-Dependent, MAPK-Independent Pathway.

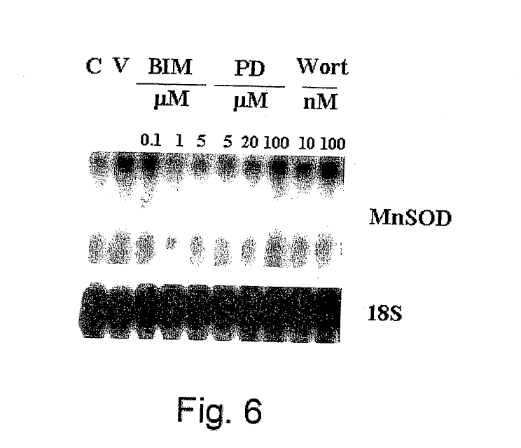

In an effort to delineate the signaling pathways responsible for mediating the effect of VEGF on MnSOD expression, endothelial cells were exposed to VEGF in the presence or absence of PD98059 or BIM. The results are shown in FIG. 6, which is a Northern blot showing the effect on VEGF-mediated induction of MnSOD mRNA production of BIM (“BIM”; 0.1, 1, 5 μM), PD98059 (“PD”; 5, 20, 100 μM) and Wortmannin (“WORT”; 10, 100 nM). BIM is an inhibitor of PKC signaling, PD98059 inhibits MAP kinase signaling, and Wortmannin is an inhibitor of PI3 kinase. Preincubation with BIM, but not PD98059, inhibited VEGF-mediated induction of MnSOD mRNA, implicating a role for PKC signaling, but not MAP kinase or PI3 kinase.

### Example 8

Endothelial Cell Proliferation is Dependent on NADPH Oxidase-Generated ROS.

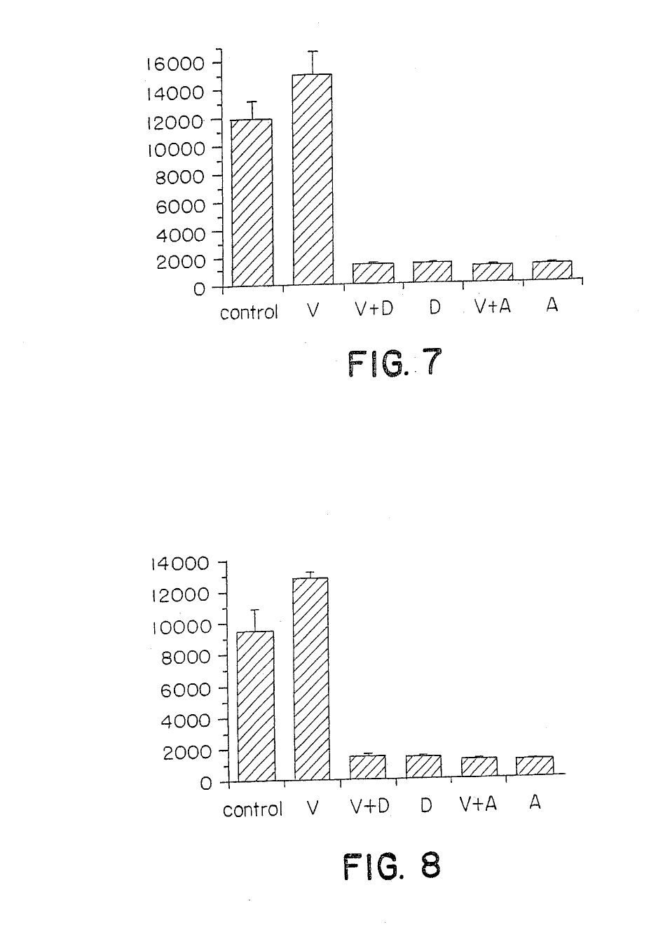

To determine whether the effect of serum or VEGF on endothelial cell proliferation might also be dependent upon ambient levels of ROS, HCAECs were grown to confluence, split into 24 wells, serum starved for eight hours and then incubated in the absence or presence of VEGF for 16 or 24 hours. All wells received 1 μCi of [³H]-thymidine at the time of treatment. After 16 or 24 hours, medium was removed, and the wells were washed 3 times with PBS. Radioactivity was extracted and thymidine incorporation measured using a scintillation counter. The results are shown in FIGS. 7 and 8, which are histograms showing thymidine incorporation (y axis) in HCAECs that have been serum starved in 0.5% FBS in the absence (control) or presence of VEGF (V) and or inhibitors (D=DPI; A=anthrone) for 16 hours (FIG. 7) or 24 hours (FIG. 8).

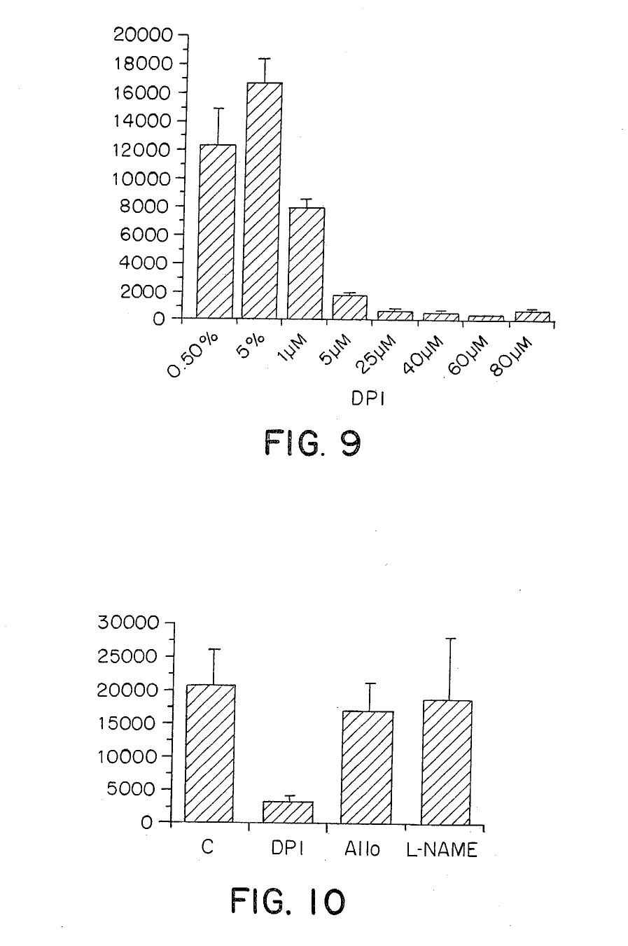

The addition of the NAPDH oxidase inhibitor DPI resulted in profound inhibition of thymidine uptake in both the serum starved and VEGF-treated cells, as did the oxygen radical scavenger, anthrone. The results of dose response studies are shown in FIG. 9, which is a histogram showing thymidine incorporation in human coronary artery endothelial cells grown in 0.5% fetal bovine serum (FBS) or 5.0% FBS in the absence (5.0% FBS) or presence of incremental doses of DPI (1, 5, 25, 40, 80 μM DPI) (x-axis). FIG. 9 shows that serum-induced thymidine incorporation was inhibited by as low as 1 μM DPI. In contrast, inhibitors of xanthine oxidase (allopurinol) or nitric oxide synthase (L-NAME) had no effect. This is shown in FIG. 10, which is a histogram showing thymidine incorporation (y-axis) in human coronary artery endothelial cells that have been grown in 10% FBS in the absence (C=control) or presence of inhibitors (DPI; Allo=allopurinol; L-NAME) (x-axis). These results show that constitutive and serum-responsive endothelial cell proliferation is dependent upon NADPH oxidase-derived ROS and that inhibitors of NADPH oxidase are potent anti-angiogenic agents.

The effect of a more specific inhibitor of NADPH oxidase, apocynin, was tested. The results are shown in FIG. 11, which is a histogram showing thymidine incorporation (y axis) in human coronary artery endothelial cells that have been grown in 0.5% FBS or 5.0% FBS in the absence (5.0% FBS) or presence of incremental doses (300 μM, 500 μM, 1 mM, 2 mM, 3 mM) apocynin (x-axis). This compound resulted in a dose-dependent inhibition of serum-induced thymidine incorporation (FIG. 11).

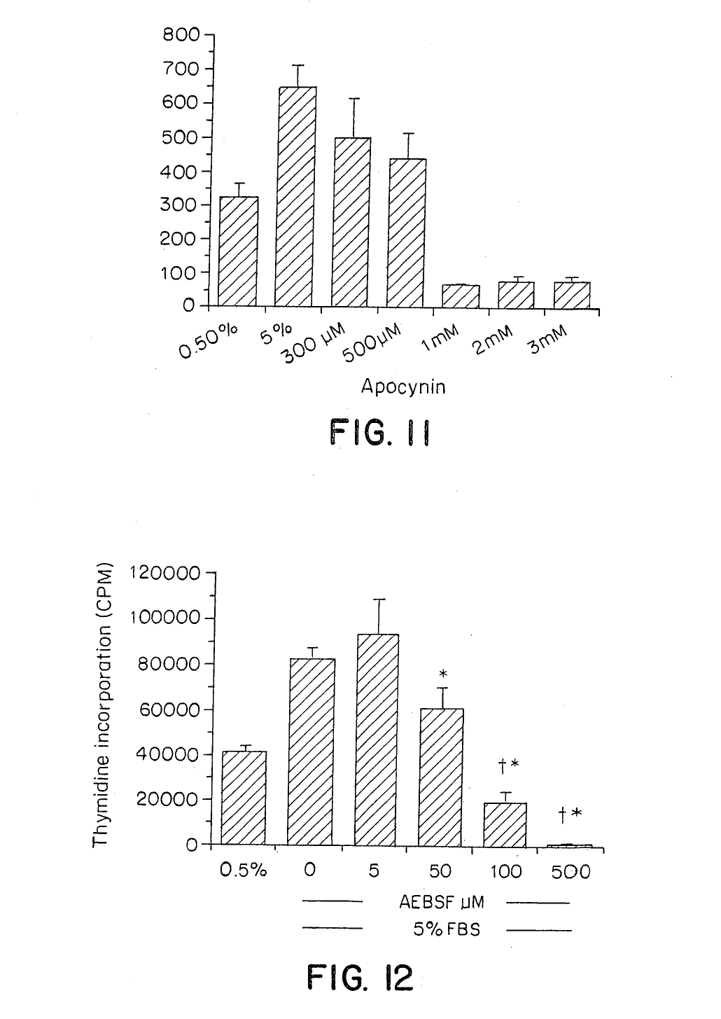

Finally, the effect of 4-(2-aminoethyl)-benzenesulfonyl fluoride (AEBSF), another inhibitor of NADPH oxidase, was tested for its effect on [³H]-thymidine uptake in HCEACs. The results are shown in FIG. 12, which is a histogram showing thymidine incorporation (cpm, x-axis) in HCAECs treated with 5% FBS and AEBSF at 0, 5, 50, 100 and 500 μM. “0.5%” represents thymidine uptake in HCAECs treated with only 0.5% FBS. AEBSF inhibited thymidine uptake in a manner that was largely dose-dependent.

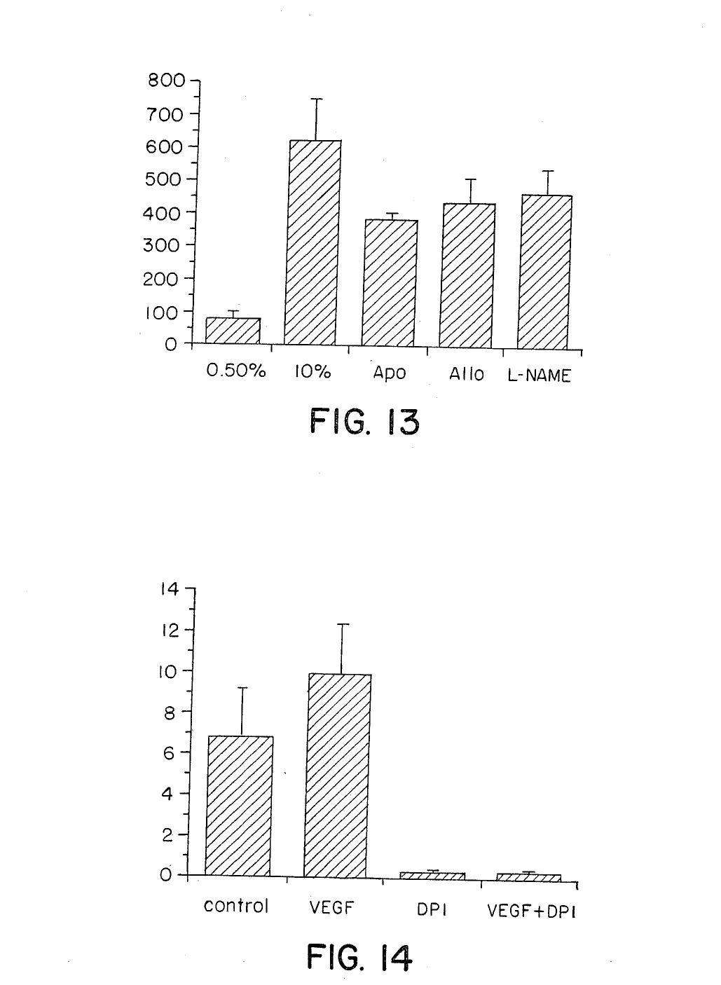

To determine whether the effect of NADPH oxidase inhibition was specific to endothelial cells, the effect of the various inhibitors on thymidine incorporation in primary murine embryonic fibroblasts was tested. The results are shown in FIG. 13, which is a histogram showing thymidine incorporation (y-axis) in mouse embryonic fibroblasts that have been grown in 0.5% FBS or 10% FBS in the absence (10% FBS) or presence of inhibitors (Apo=apocynin; allo=allopurinol; L-NAME) for 16 hours. Apocynin failed to reduce incorporation below baseline. These results suggest that the proliferation of non-endothelial cells may not be dependent on ambient levels of NADPH oxidase-generated ROS.

### Example 9

Inhibition of NADPH Oxidase Inhibits Migration of Endothelial Cells.

The capacity of NADPH oxidase inhibitors to block migration of human coronary artery endothelial cells was tested. In these studies, VEGF-induced chemotaxis was tested with the Boyden chamber assay. Human coronary artery endothelial cells were serum starved in 0.5% FBS overnight. 25,000 cells were seeded into the upper chamber in the absence or presence of DPI, while media containing 0.5% FBS and 40 ng/ml was placed into the lower chamber. The chamber was incubated for 4 hours at 37° C., at which time the polycarbonate filters were harvested and counted for migrating cells. The results are shown in FIG. 14, which is a histogram showing the migration in terms of cell count (y-axis) of human coronary artery endothelial cells in a Boyden chamber. The cells were serum starved in 0.5% FBS and treated with either VEGF, DPI or VEGF+DPI (x-axis). The addition of 25 μM DPI results in profound inhibition of migration.

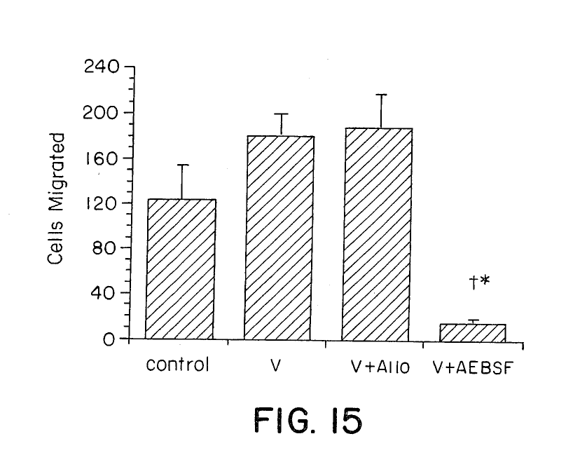

The effect of AEBSF on VEGF-induced migration was also studied. HCAECs were treated as above and treated by either VEGF, VEGF+100 μM allopurinol, or VEGF+250 μM AEBSF. The results are shown in FIG. 15, which is a histogram showing migration in terms of cell count (y-axis) when treated (x-axis). The NADPH oxidase inhibitor AEBSF abrograted the VEGF-mediated chemotaxis of HCAECs.

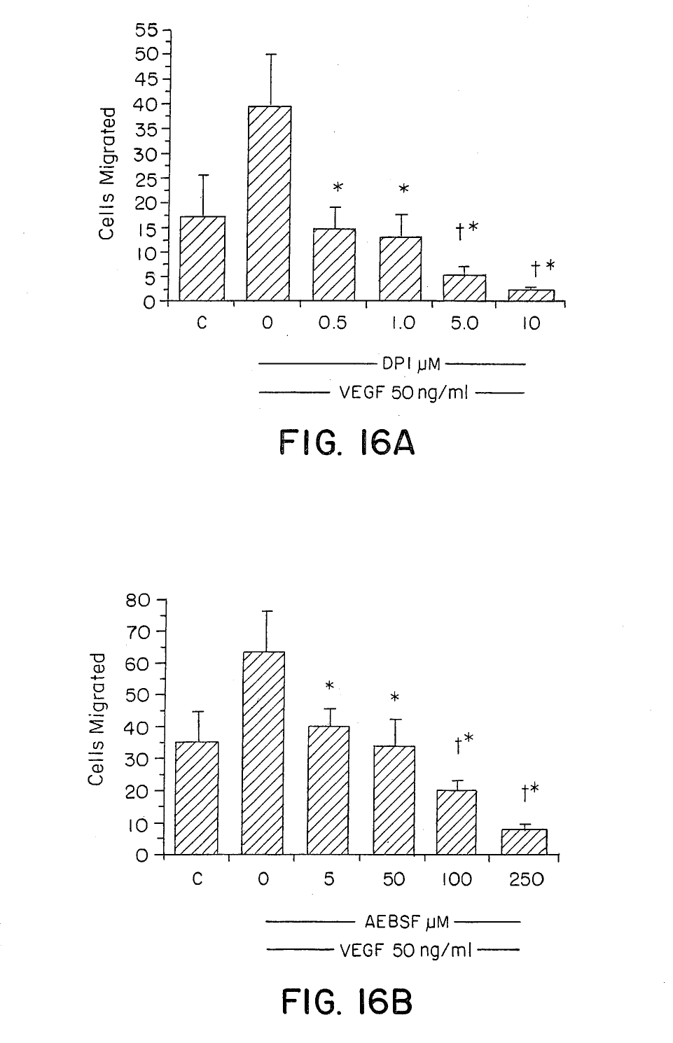

The inhibition of migration was found to be dose-dependent, as is shown in FIGS. 16A and 16B. These are histograms showing VEGF-mediated chemotaxis in control HCAECs (“C”), or HCAECs treated with 50 ng/ml VEGF in the absence or presence of increasing doses of DPI (0.5, 1.0, 5.0 or 10 μM; FIG. 16A) or AEBSF (5, 50, 100 or 250 μM; FIG. 16B).

All references, patents and patent applications cited are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A method of inhibiting angiogenesis in a tissue, the method comprising contacting the tissue with an inhibitor of NADPH oxidase.

2. The method of claim 1, wherein the inhibitor of NADPH oxidase is a chemical inhibitor.

3. The method of claim 2, wherein the inhibitor of NADPH oxidase is selected from the group consisting of: diphenyleneiodonium (DPI), apocynin, 4-(2-aminoethyl)-benzenesulfonyl fluoride (AEBSF).

4. The method of claim 1, wherein the inhibitor of NADPH oxidase is an enzyme.

5. The method of claim 4, wherein the inhibitor of NADPH oxidase is a superoxide dismutase (SOD).

6. The method of claim 5, wherein the SOD is selected from the group consisting of: mitochondrial SOD (MnSOD), cytosolic Cu, Zn-SOD and extracellular Cu, Zn-SOD.

7. A method of inhibiting angiogenesis in a tissue, the method comprising inhibiting the production of reactive oxygen species (ROS) in the tissue.

8. The method of claim 7, wherein the inhibition of the production of reactive oxygen species (ROS) is accomplished by contacting the tissue with an inhibitor of NADPH oxidase.

9. The method of claim 8, wherein the inhibitor of NADPH oxidase is a chemical inhibitor.

10. The method of claim 9, wherein the inhibitor of NADPH oxidase is selected from the group consisting of: diphenyleneiodonium (DPI), apocynin, 4-(2-aminoethyl)-benzenesulfonyl fluoride (AEBSF).

11. The method of claim 7, wherein the inhibitor of NADPH oxidase is an enzyme.

12. The method of claim 11, wherein the inhibitor of NADPH oxidase is a superoxide dismutase (SOD).

13. The method of claim 12, wherein the SOD is selected from the group consisting of: mitochondrial SOD (MnSOD), cytosolic Cu, Zn-SOD and extracellular Cu, Zn-SOD.

14. The method of claim 7, wherein the inhibition of the production of reactive oxygen species (ROS) is accomplished by contacting the tissue with an inhibitor of superoxide dismutase (SOD) mRNA induction.

15. The method of claim 14, wherein the SOD is mitochondrial SOD (MnSOD).

16. A method of inhibiting angiogenesis in a tissue, the method comprising inhibiting induction of mRNA of a superoxide dismutase (SOD) in the tissue.

17. The method of claim 16, wherein the SOD is selected from the group consisting of: mitochondrial SOD (MnSOD), cytosolic Cu, Zn-SOD and extracellular Cu, Zn-SOD.

18. The method of claim 17, wherein the SOD is mitochondrial SOD (MnSOD).

19. A method of inhibiting endothelial cell migration in a tissue, the method comprising contacting the tissue with an inhibitor of NADPH oxidase.

20. The method of claim 19, wherein the inhibitor of NADPH oxidase is a chemical inhibitor.

21. The method of claim 20, wherein the inhibitor of NADPH oxidase is selected from the group consisting of: diphenyleneiodonium (DPI), apocynin, 4-(2-aminoethyl)-benzenesulfonyl fluoride (AEBSF).

22. The method of any of claim 19, wherein the inhibitor of NADPH oxidase is an enzyme.

23. The method of claim 20, wherein the inhibitor of NADPH oxidase is a superoxide dismutase (SOD).

24. The method of claim 23, wherein the SOD is selected from the group consisting of: mitochondrial SOD (MnSOD), cytosolic Cu, Zn-SOD and extracellular Cu, Zn-SOD.

25. A method of inhibiting endothelial cell migration in a tissue, the method comprising inhibiting the production of reactive oxygen species (ROS) in the tissue.

26. The method of claim 25, wherein the inhibition of the production of reactive oxygen species (ROS) is accomplished by contacting the tissue with an inhibitor of NADPH oxidase.

27. The method of claim 25, wherein the inhibitor of NADPH oxidase is a chemical inhibitor.

28. The method of claim 27, wherein the inhibitor of NADPH oxidase is selected from the group consisting of: diphenyleneiodonium (DPI), apocynin, 4-(2-aminoethyl)-benzenesulfonyl fluoride (AEBSF).

29. The method of claim 25, wherein the inhibitor of NADPH oxidase is an enzyme.

30. The method of claim 29, wherein the inhibitor of NADPH oxidase is a superoxide dismutase (SOD).

31. The method of claim 30, wherein the SOD is selected from the group consisting of: mitochondrial SOD (MnSOD), cytosolic Cu, Zn-SOD and extracellular Cu, Zn-SOD.

32. The method of claim 25, wherein the inhibition of the production of reactive oxygen species (ROS) is accomplished by contacting the tissue with an inhibitor of superoxide dismutase (SOD) mRNA induction.

33. The method of claim 32, wherein the SOD is mitochondrial SOD (MnSOD).

34. A method of inhibiting endothelial cell migration in a tissue, the method comprising inhibiting induction of mRNA of a superoxide dismutase (SOD) in the tissue.

35. The method of claim 34, wherein the SOD is selected from the group consisting of: mitochondrial SOD (MnSOD), cytosolic Cu, Zn-SOD and extracellular Cu, Zn-SOD.

36. The method of claim 35, wherein the SOD is mitochondrial SOD (MnSOD).

37. A method of inhibiting endothelial cell proliferation in a tissue, the method comprising contacting the tissue with an inhibitor of NADPH oxidase.

38. The method of claim 37, wherein the inhibitor of NADPH oxidase is a chemical inhibitor.

39. The method of claim 38, wherein the inhibitor of NADPH oxidase is selected from the group consisting of: diphenyleneiodonium (DPI), apocynin, 4-(2-aminoethyl)-benzenesulfonyl fluoride (AEBSF).

40. The method of claim 37, wherein the inhibitor of NADPH oxidase is an enzyme.

41. The method of claim 40, wherein the inhibitor of NADPH oxidase is a superoxide dismutase (SOD).

42. The method of claim 41, wherein the SOD is selected from the group consisting of: mitochondrial SOD (MnSOD), cytosolic Cu, Zn-SOD and extracellular Cu, Zn-SOD.

43. A method of inhibiting endothelial cell proliferation in a tissue, the method comprising inhibiting the production of reactive oxygen species (ROS) in the tissue.

44. The method of claim 43, wherein the inhibition of the production of reactive oxygen species (ROS) is accomplished by contacting the tissue with an inhibitor of NADPH oxidase.

45. The method of claim 43, wherein the inhibitor of NADPH oxidase is a chemical inhibitor.

46. The method of claim 45, wherein the inhibitor of NADPH oxidase is selected from the group consisting of: diphenyleneiodonium (DPI), apocynin, 4-(2-aminoethyl)-benzenesulfonyl fluoride (AEBSF).

47. The method of claim 43, wherein the inhibitor of NADPH oxidase is an enzyme.

48. The method of claim 47, wherein the inhibitor of NADPH oxidase is a superoxide dismutase (SOD).

49. The method of claim 48, wherein the SOD is selected from the group consisting of: mitochondrial SOD (MnSOD), cytosolic Cu, Zn-SOD and extracellular Cu, Zn-SOD.

50. The method of claim 43, wherein the inhibition of the production of reactive oxygen species (ROS) is accomplished by contacting the tissue with an inhibitor of superoxide dismutase (SOD) mRNA induction.

51. The method of claim 50, wherein the SOD is mitochondrial SOD (MnSOD).

52. A method of inhibiting endothelial cell proliferation in a tissue, the method comprising inhibiting induction of mRNA of a superoxide dismutase (SOD) in the tissue.

53. The method of claim 52, wherein the SOD is selected from the group consisting of: mitochondrial SOD (MnSOD), cytosolic Cu, Zn-SOD and extracellular Cu, Zn-SOD.

54. The method of claim 53, wherein the SOD is mitochondrial SOD (MnSOD).

55. A composition comprising an inhibitor of NADPH oxidase, the composition having one or more properties selected from the group consisting of: the ability to inhibit angiogenesis, the ability to inhibit endothelial cell migration, the ability to inhibit endothelial cell proliferation and the ability to inhibit VEGF-mediated angiogenesis; wherein the composition optionally further comprises a pharmaceutically compatible carrier.

56. The composition of claim 55, wherein the inhibitor is a chemical inhibitor.

57. The composition of claim 56, wherein the inhibitor is selected from the group consisting of: diphenyleneiodonium (DPI), apocynin, 4-(2-aminoethyl)-benzenesulfonyl fluoride (AEBSF).

58. The composition of claim 55, wherein the inhibitor of NADPH oxidase is an enzyme.

59. The composition of claim 58, wherein the inhibitor of NADPH oxidase is a superoxide dismutase (SOD).

60. A method of treating a disorder involving inhibiting angiogenesis in a tissue, comprising administering the composition of claim 55.

61. A method of treating a disorder involving inhibiting endothelial cell migration in a tissue, comprising administering the composition of claim 55.

62. A method of treating a disorder involving inhibiting endothelial cell proliferation in a tissue, comprising administering the composition of claim 55.

63. A method of treating a disorder involving inhibiting VEGF-mediated angiogenesis in a tissue, comprising administering the composition of claim 55.

64. The method of claim 55, wherein the disorder is selected from the group consisting of: angiogenesis-dependent cancers, benign tumors, rheumatoid arthritis, psoriasis, ocular angiogenesis diseases, Osler-Webber Syndrome, myocardial angiogenesis, plaque neovascularization, telangiectasia, hemophiliac joints, angiofibroma, wound granulation, intestinal adhesions, atherosclerosis, scleroderma, hypertrophic scars, cat scratch disease, Heliobacter pylori ulcers, dialysis graft vascular access stenosis, contraception and obesity.

65. The method of claim 64, wherein the disorder is tumor growth.

66. The method of claim 64, wherein the disease is cancer.

67. A composition comprising an inhibitor of ROS production, the composition having one or more properties selected from the group consisting of: the ability to inhibit angiogenesis, the ability to inhibit endothelial cell migration, the ability to inhibit endothelial cell proliferation and the ability to inhibit VEGF-mediated angiogenesis; wherein the composition optionally further comprises a pharmaceutically compatible carrier.

68. The composition of claim 67, wherein the inhibitor is a chemical inhibitor.

69. The composition of claim 68, wherein the inhibitor is selected from the group consisting of: diphenyleneiodonium (DPI), apocynin, 4-(2-aminoethyl)-benzenesulfonyl fluoride (AEBSF).

70. A method of treating a disorder involving inhibiting angiogenesis in a tissue comprising administering the composition of claim 67.

71. A method of treating a disorder involving inhibiting endothelial cell migration in a tissue, comprising administering the composition of claim 67.

72. A method of treating a disorder involving inhibiting endothelial cell proliferation in a tissue, comprising administering the composition of claim 67.

73. A method of treating a disorder involving inhibiting VEGF-mediated angiogenesis in a tissue, comprising administering the composition of claim 67.

74. The method of claim 67, wherein the disorder is selected from the group consisting of: angiogenesis-dependent cancers, benign tumors, rheumatoid arthritis, psoriasis, ocular angiogenesis diseases, Osler-Webber Syndrome, myocardial angiogenesis, plaque neovascularization, telangiectasia, hemophiliac joints, angiofibroma, wound granulation, intestinal adhesions, atherosclerosis, scleroderma, hypertrophic scars, cat scratch disease, Heliobacter pylori ulcers, dialysis graft vascular access stenosis, contraception and obesity.

75. The method of claim 74, wherein the disorder is tumor growth.

76. The method of claim 74, wherein the disease is cancer.

77. A composition comprising an inhibitor of induction of mRNA of a SOD, the composition having one or more properties selected from the group consisting of: the ability to inhibit angiogenesis, the ability to inhibit endothelial cell migration, the ability to inhibit endothelial cell proliferation and the ability to inhibit VEGF-mediated angiogenesis; wherein the composition optionally further comprises a pharmaceutically compatible carrier.

78. The composition of claim 77, wherein the inhibitor is a chemical inhibitor.

79. The composition of claim 78, wherein the inhibitor is selected from the group consisting of: diphenyleneiodonium (DPI), apocynin, 4-(2-aminoethyl)-benzenesulfonyl fluoride (AEBSF).

80. The composition of claim 77, wherein the SOD is mitochondrial SOD (MnSOD).

81. A method of treating a disorder involving inhibiting angiogenesis in a tissue comprising administering the composition of claim 77.

82. A method of treating a disorder involving inhibiting endothelial cell migration in a tissue, comprising administering the composition of claim 77.

83. A method of treating a disorder involving inhibiting endothelial cell proliferation in a tissue, comprising administering the composition of claim 77.

84. A method of treating a disorder involving inhibiting VEGF-mediated angiogenesis in a tissue, comprising administering the composition of claim 77.

85. The method of claim 77, wherein the disorder is selected from the group consisting of: angiogenesis-dependent cancers, benign tumors, rheumatoid arthritis, psoriasis, ocular angiogenesis diseases, Osler-Webber Syndrome, myocardial angiogenesis, plaque neovascularization, telangiectasia, hemophiliac joints, angiofibroma, wound granulation, intestinal adhesions, atherosclerosis, scleroderma, hypertrophic scars, cat scratch disease, Heliobacter pylori ulcers, dialysis graft vascular access stenosis, contraception and obesity.

86. The method of claim 85, wherein the disorder is tumor growth.

87. The method of claim 85, wherein the disease is cancer.
